(19) United States
(12) Patent Application Publication
(10) Pub. No.: US 2022/0370520 A1
(43) Pub. Date: Nov. 24, 2022

(54) COMPOSITIONS BASED ON BACTERIAL STRAINS AND BERRY EXTRACTS AND THEIR USE AS ANTI-INFLAMMATORIES

(71) Applicant: SOFAR S.P.A., Trezzano Rosa (MI) (IT)

(72) Inventors: Andrea BIFFI, Urgnano (IT); Walter FIORE, Atrani (IT)

(21) Appl. No.: 17/761,920

(22) PCT Filed: Sep. 21, 2020
(86) PCT No.: PCT/IB2020/058777

§ 371 Date: Mar. 18, 2022

(30) Foreign Application Priority Data
Sep. 20, 2019 (IT) 102019000016850

(51) Int. Cl. A61K 35/745; A61K 35/747; A61K 36/45; A61P 29/00

(52) U.S. Cl. CPC A61K 35/745; A61K 35/747; A61K 36/45; A61P 29/00

(57) ABSTRACT

A composition comprising a mixture comprising or, alternatively, consisting of: at least one bacterial strain selected from a group comprising or, alternatively, consisting of bacterial strains belonging to the species Lactobacillus paracasei, Lactobacillus rhamnosus, Bifidobacterium bifidum and Bifidobacterium animalis subsp. Lactis, and preferably, at least one extract of at least one species of berries comprising a polyphenol fraction of said berries and a related method wherein said composition is used as an immunomodulatory and anti-inflammatory agent are disclosed.

The present invention relates to compositions A comprising a mixture comprising or, alternatively, consisting of at least two bacterial strains selected from a group comprising or, alternatively, consisting of bacterial strains belonging to the species Lactobacillus paracasei, Lactobacillus rhamnosus, Bifidobacterium bifidum and Bifidobacterium animalis subsp. lactis. Alternatively, the present invention relates to compositions B comprising a mixture comprising or, alternatively, consisting of: at least one bacterial strain selected from a group comprising or, alternatively, consisting of bacterial strains belonging to the species Lactobacillus paracasei, Lactobacillus rhamnosus, Bifidobacterium bifidum and Bifidobacterium animalis subsp. lactis, and at least one extract of at least one species of berries comprising a polyphenol fraction of said berries. Furthermore, the present invention relates to said compositions A and compositions B for use as immunomodulatory and anti-inflammatory agents.

Over the last few decades, the scientific community has conducted studies on the modification of the gut microbiota with the aim of obtaining effects on the health of a subject. It is well known that gut microbiota is a key factor contributing to digestive processes, production of vitamins, transformation of bile acids, generating a multitude of bioactive compounds from the food components. For example, short-chain fatty acids are produced by the fermentation of fibres, linoleic acids conjugated by linoleic acid, enterodiol and lignan enterolactone, all linked to antitumor, anti-inflammatory and other health-promoting effects. Beneficial bacteria in the gut microbiota also play an important role in immunity through the modulation of local and systemic immunity responses and they can prevent the growth of pathogenic bacteria through competition mechanisms known as barrier effect. Although the composition of gut microbial species is extremely variable from one person to another, it is relatively constant for each individual adult, and it is mostly determined by genetic factors and gut colonisation in the early stages of life. However, the composition thereof may be significantly affected by various factors, such as diet and the intake of probiotic products or prebiotic products or live biotherapeutics products (in short LBP, pharmaceutical products based on viable bacterial strains).

Therefore, the scientific community's interest in having compositions capable of providing positive effects by interacting on the gut microbiota, in particular compositions capable of exerting anti-inflammatory effects by modulating the response of the immune system to inflammatory stimuli remains high.

Following an intense research and development phase, the Applicant found that compositions comprising specific mixtures of at least two bacterial strains and/or compositions comprising at least one or more bacterial strains and an extract of at least one species of berries comprising the polyphenolic portion of said berries are capable of positively modulating the responses of the immune system and exerting an anti-inflammatory action as described in detail in the present description and in the attached claims.

In the context of the present invention, the term “berries” is used to indicate the so-called “wild berries”, as a category of small fleshy, sweet or sour edible fruits, whose plants grow in the particular humid climate and acid soil of the undergrowth, in semi-shadow conditions and cold climate.

In the context of the present invention, the terms “berries” and “wild berries” are synonyms.

In the context of the present invention, the species of “berries” or “wild berries” comprise at least one of the following examples: blueberry or European blueberry (Vaccinium cyanococcus or Vaccinium myrtillus or Vaccinium angustifolium), oxycoccus or cranberry (Vaccinium oxycoccos or Vaccinium macrocarpon), wild strawberry or strawberry (Fragaria vesca or Fragaria spp. or Fragaria ananassa), elderberry (Sambucus nigra), cowberry (Vaccinium vitis-idaea), blackberry (Rubus ulmifolius), red raspberry (Rubus idaeus), black raspberry (Rubus leucodermis or Rubus occidentalis), black currant (Ribes nigrum), red currant (Ribes rubrum), black mulberry (Morus nigra), black mulberry (Morus rubra), white mulberry (Morus alba), cornelian cherry (Cornus mas), gooseberry (Ribes uva-crispa), barberry (Berberis vulgaris), Amelanchier ovalis, Amelanchier canadensis, mahaleb cherry (Prunus mahaleb), sour cherries and black cherries (Prunus cerasus), strawberry tree (Arbutus unedo).

In particular,
blueberry (European blueberry or wild blueberry) is the fruit (blue or purple berries) of perennial plants classified under the genus Vaccinium. The American blueberry is classified under the species Vaccinium cyanoccus (Rydb.), whereas the bilberry is classified under the species Vaccinium myrtillus (L., 1753); furthermore, there exists Vaccinium angustifolium (Aiton, 1789), commonly known as wild blueberry, originating in eastern and central Canada and in the north-eastern part of the United States; in the context of the present invention, the term blueberry is preferably used to indicate the species Vaccinium myrtillus and Vaccinium angustifolium;
cranberry (oxycoccus or bearberry or American cranberry) is the fruit of a group of evergreen dwarf shrubs or final vines classified under the species oxycoccus of the genus Vaccinium. In Great Britain, cranberry refers to the autochthonous species Vaccinium oxycoccos (L., 1753) or oxycoccus, whereas in North America cranberry refers to Vaccinium macrocarpon (Aiton 1789) or bearberry or American cranberry; in the context of the present invention, the term cranberry is preferably used to indicate the species Vaccinium macrocarpon;
strawberries (wild strawberry or strawberry) are fruits of a herbaceous plant classified under the species Fragaria vesca (L., 1753) or Fragaria spp. or Fragaria anananassa (Duchesne) of the genus Fragaria of the family Rosaceae;
elderberry (black elderberry) is the fruit of a plant classified under the species Sambucus nigra (L., 1753) of the genus Sambucus of the family Adoxaceae;
black raspberry is the fruit of three species of plants belonging to the genus Rubus: Rubus leucodermis (Dougal. Ex Torr. & A. Gray 1840) originating in western North America, Rubus occidentalis (L., 1753) originating in eastern North America, and Rubus coreanus (Miq. 1867), also known as black Korean raspberry originating in Korea, Japan and China;
red raspberry is the fruit of a plant classified under the species Rubus idaeus (L., 1753) of the genus Rubus of the family Rosaceae.

In the context of the present invention, reference will be made to the aforementioned species of berries using the Italian or English names interchangeably.

Since the end of the 20th century there has been a high interest by the scientific community for the beneficial effects of said berries. Berries are generally known as nutritive foods, given that they contain large amounts of water-soluble vitamins, minerals (potassium, manganese, zinc) and fibres. However, it is hypothesised that the polyphenols contained therein are the main component of the benefits attributable thereto, such as for example antioxidant and anti-inflammatory properties.

Berries are rich in polyphenols, such as for example anthocyanins, anthocyanidins and/or proanthocyanidins.

Proanthocyanidins are a class of polyphenols present in numerous varieties of botanical species. They are chemically oligomeric repeats of flavonoids, such as for example oligomeric repeats of catechin and epicatechin and their esters of gallic acid.

Anthocyanins (or anthocyans) belong to the family of flavonoids and they derive from their respective aglycones (anthocyanidins), from which they differ by the addition of one or more glycoside groups (sugars).

In the last two decades, a considerable number of studies have been implemented to determine the potential health benefits of said berries. The high antioxidant power of berries can, in part, explain their protective activity against degenerative processes linked to oxidative stress and to the presence of reactive oxygen species, which are also the main reason for the protective activity at the cardiovascular level and the anticarcinogenic activity attributed in general to the presence of polyphenols in foods. Furthermore, besides the significant antioxidant effects, phenolic acids and resveratrol (polyphenol) contained in berries account for considerable metabolic effects. The considerable presence of anthocyanidins (polyphenols) also contributes to a specific anti-inflammatory action at the level of the locomotor system (muscular and skeletal system), digestive system, urogenital system (urinary system and genital system), respiratory system, integumentary system, immune system and circulatory system. Lastly, the various berries have shown specific antibacterial and prebiotic activities in terms of prevention of bacterial adhesion to the uroepithelial surface, inhibition of biofilm, modification of gene expression and membrane structure, modification of the gut microbiota.

The mixtures and compositions of the invention (compositions (A) of the invention and compositions (B) of the invention, as defined hereinafter) reveal to have anti-inflammatory and immunomodulatory effects (IL-10:IL-12 ratio>>1) and they do not show any significant adverse effects, therefore they can be administered to any type of subject, including pregnant women, paediatric and elderly subjects. Furthermore, the mixtures and the compositions of the invention are effective, easy to prepare and cost-effective to produce.

These and other objects which will be clearer from the detailed description that follows, are achieved by the bacterial strain, by the compositions and by the mixtures of the present invention thanks to the technical characteristics present in the description and claimed in the attached claims.

### BRIEF DESCRIPTION OF THE FIGURES

FIG. 1a, 1b, 1c: response of cytokines IL12, TNF-α and IL10 after stimulation with single bacterial strains of group (I.1), in the presence or absence of LPS (lipopolysaccharide, inflammatory stimulus), [*]=p<0.05, [+]=p<0.01, [$]=p<0.001;

FIG. 2a, 2b, 2c: response of cytokines IL12, TNF-α and IL10 after stimulation with mixtures of bacterial strains of group (I.1), [+]=p<0.01; data expressed as pg/mL;

FIG. 3: Response of cytokines IL12, TNF-α and IL10 after stimulation with mixtures comprising a bacterial strain of group (I.1) and an extract of berries comprising the polyphenol fraction; C: control (BMDCs stimulated with RPMI medium only).

FIG. 4: response of cytokines IL12, TNF-α and IL10 after stimulation with mixtures of at least 1 or 2 bacterial strains of group (I.1) and an extract of berries comprising the polyphenol fraction;

FIG. 4a: response of cytokines IL12, TNF-α and IL10 after stimulation with mixtures of at least 2 bacterial strains of group (I.1) and a berry extract comprising the polyphenol fraction; C: control (BMDCs stimulated with RPMI medium only), [*]=p<0.05, [+]=p<0.01, [$]=p<0.001.

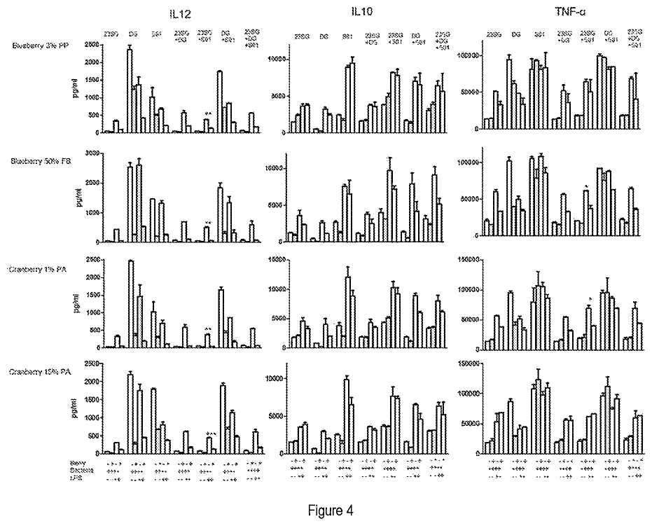

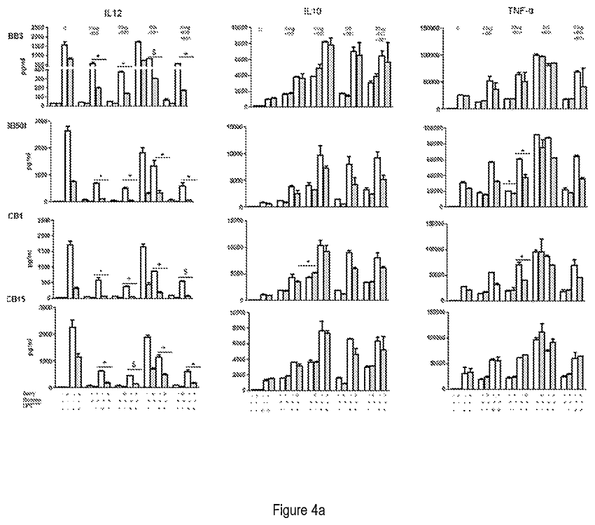

In FIGS. 4 and 4a the polyphenols extracted from the berries are used at a concentration of 50 μg/mL; the combinations of bacteria are used at a final MOI (multiplicity of infection) of 5.

### DETAILED DESCRIPTION OF THE INVENTION

Forming an object of the present invention is a composition (A) (in short, composition (A) of the invention) comprising a mixture (A) (in short, mixture (A) of the invention) comprising or, alternatively, consisting of at least two bacterial strain selected from the group (I) comprising or, alternatively, consisting of bacterial strains belonging to the species Lactobacillus paracasei, Lactobacillus rhamnosus, Bifidobacterium bifidum and Bifidobacterium animalis subsp. lactis, wherein said at least two bacterial strains are selected from the group (I.i) comprising or, alternatively, consisting of: Lactobacillus paracasei DG® (CNCM I-1572), Lactobacillus paracasei LPC-S01™ (DSM 26760), Bifidobacterium bifidum MIMBb23sg (DSM 32708), Lactobacillus paracasei CF3 (DSM 32353), Lactobacillus rhamnosus GG (DSM 53103), Bifidobacterium animalis subsp. lactis Bb12 (DSM 15954), and wherein, optionally, said composition (A) comprises at least one food or pharmacological grade additive and/or excipient.

A bacterial strain identified as Lactobacillus paracasei DG® (trademark registered by SOFAR S.p.A.) was deposited by SOFAR S.p.A. at the National Collection of Cultures of Microorganisms of the Pasteur Institute in Paris under access number CNCM I-1572 on 5 May 1995 by SOFAR S.p.A. (in short, DG® or L. paracasei DG® CNCM I-1572). The strain was initially named Lactobacillus casei DG® sub. casei CNCM I-1572; it was subsequently reclassified as Lactobacillus paracasei DG® CNCM I-1572. It should be observed that it is still and exclusively the same bacterial strain irrespective of the name Lactobacillus casei DG® CNCM I-1572 or Lactobacillus paracasei DG® CNCM I-1572.

A bacterial strain identified as Lactobacillus paracasei LPC-S01™ was deposited at Deutsche Sammlung von Mikroorganismen und Zellkulturen GmbH (DSMZ) under access number DSM 26760 on 15 May 2017 by SOFAR S.p.A. (date of application for conversion of the deposit into a deposit according to the Budapest Treaty; date of original deposit: 11 Jan. 2013) (in short, LPC-S01™ or L. paracasei LPC-S01™ DSM 26760). It should be observed that it is still and exclusively the same bacterial strain irrespective of the name used by the Applicant Lactobacillus paracasei S01 DSM 26760 or Lactobacillus paracasei LPC-S01™ DSM 26760.

A bacterial strain identified as Bifidobacterium bifidum MIMBb23sg (or, alternatively, MIMBb23SG), alternatively named B. bifidum BbflBLPC-S01™ or B. bifidum BbflBS01, was deposited at Deutsche Sammlung von Mikroorganismen und Zellkulturen GmbH (DSMZ) under deposit number DSM 32708 on 4 Dec. 2017 by SOFAR S.p.A. (in short, 23sg or B. bifidum MIMBb23sg DSM 32708 or B. bifidum BbflBLPC-S01™ DSM 32708). It should be observed that it is still and exclusively the same bacterial strain irrespective of the internal name MIMBb23sg or BbflBLPC-S01™ or BbflBS01, used by the Applicant.

A bacterial strain identified as Lactobacillus paracasei CF3 was deposited at Deutsche Sammlung von Mikroorganismen und Zellkulturen GmbH (DSMZ) under access number DSM 32353 on 4 Aug. 2016 by SOFAR S.p.A. (in short, CF3 or L. paracasei CF3 DSM 32353).

A bacterial strain identified as Lactobacillus rhamnosus GG was deposited at Deutsche Sammlung von Mikroorganismen und Zellkulturen GmbH (DSMZ) under access number DSM 53103 (in short, GG or L. paracasei GG DSM 53103).

A bacterial strain identified as Bifidobacterium animalis subsp. lactis Bb12 was deposited at Deutsche Sammlung von Mikroorganismen und Zellkulturen GmbH (DSMZ) under deposit number DSM 15954 (in short, Bb12 or B. animalis subsp. lactis. Bb12 DSM 15954).

All the strains mentioned in the present invention were deposited according to the Budapest Treaty.

In the composition (A) of the invention, the mixture (A) of the invention may comprise 2, 3, 4, 5 or 6 bacterial strains selected from group (I.i) as defined in the present invention.

Advantageously, in said mixture (B) comprising 2, 3, 4, 5 or 6 bacterial strains selected from group (I.i), the bacterial strains are at a CFU ratio with respect to each other of about 1:1 or 1:1:1 or 1:1:1:1 or 1:1:1:1:1 or 1:1:1:1:1:1.

In an embodiment of the composition (A) of the invention, the mixture (A) comprises or, alternatively, consists of a bacterial strain B. bifidum MIMBb23sg DSM 32708 and at least one bacterial strain selected from the group (I.ii) comprising or, alternatively, consisting of: L. paracasei DG® (CNCM I-1572), L. paracasei LPC-S01™ (DSM 26760), L. paracasei CF3 (DSM 32353), L. rhamnosus GG (DSM 53103), B. animalis subsp. lactis Bb12 (DSM 15954), and mixtures thereof.

In a preferred embodiment of the composition (A) of the invention, the mixture (A) comprises or, alternatively, consists of B. bifidum MIMBb23sg DSM 32708 and L. paracasei LPC-S01™ (DSM 26760). In a preferred embodiment of the composition (A) of the invention, the mixture (A) comprises or, alternatively, consists of B. bifidum MIMBb23sg (DSM 32708) and L. paracasei DG® (CNCM I-1572).

In a preferred embodiment of the composition (A) of the invention, the mixture (A) comprises or, alternatively, consists of B. bifidum MIMBb23sg DSM 32708, L. paracasei LPC-S01™ (DSM 26760) and of at least one bacterial strain selected from the group (I.iii) comprising or, alternatively, consisting of: L. paracasei DG® (CNCM I-1572), L. paracasei CF3 (DSM 32353), L. rhamnosus GG (DSM 53103), B. animalis subsp. lactis Bb12 (DSM 15954), and mixtures thereof.

In a preferred embodiment of the composition (A) of the invention, the mixture (A) comprises or, alternatively, consists of B. bifidum MIMBb23sg DSM 32708, L. paracasei DG® (CNCM I-1572) and of at least one bacterial strain selected from the group (I.iii) comprising or, alternatively, consisting of: L. paracasei LPC-S01™ (DSM 26760), L. paracasei CF3 (DSM 32353), L. rhamnosus GG (DSM 53103), B. animalis subsp. lactis Bb12 (DSM 15954), and mixtures thereof.

In a preferred embodiment of the composition (A) of the invention, the mixture (A) comprises or, alternatively, consists of B. bifidum MIMBb23sg (DSM 32708) and L. paracasei LPC-S01™ (DSM 26760) and L. paracasei DG® (CNCM I-1572).

For example, the composition (A) of the invention may comprise the mixture (A) comprising or, alternatively, consisting of B. bifidum MIMBb23sg DSM 32708, L. paracasei LPC-S01™ (DSM 26760), L. paracasei DG® (CNCM I-1572) and of at least one bacterial strain selected from the group comprising or, alternatively, consisting of: L. paracasei LPC-S01™ (DSM 26760), L. paracasei CF3 (DSM 32353), L. rhamnosus GG (DSM 53103), B. animalis subsp. lactis Bb12 (DSM 15954), and mixtures thereof.

Forming an object of the present invention is a composition (B) (in short, composition (B) of the invention) comprising a mixture (B) (in short, mixture (B) of the invention) comprising or, alternatively, consisting of:
at least one or a mixture of bacterial strains selected from group (I) comprising or, alternatively, consisting of bacterial strains belonging to the species Lactobacillus paracasei, Lactobacillus rhamnosus, Bifidobacterium bifidum and Bifidobacterium animalis subsp. Lactis, and
at least one extract of at least one species of berries comprising or, alternatively, consisting of the polyphenolic fraction of said berries (in short, extract of the invention); and wherein, optionally, said composition (B) comprises at least one food or pharmacological grade additive and/or excipient.

Said polyphenolic fraction of said berries is preferably obtained according to the extraction method of the invention described hereinafter or, alternatively, according to methods and equipment known to the man skilled in the art.

In the context of the present invention, said polyphenolic fraction of said extract of said at least one species of berries comprises at least one or more proanthocyanidins (of type A and/or of type B) and/or anthocyanins or anthocyanidins (e.g. malvidin, or peonidin). The terms “anthocyanins” and “anthocyans” are synonyms, used in the context of the present invention interchangeably.

Anthocyans (or anthocyanins) are among the most important polyphenolic compounds present in the berries of the present invention (for example, cranberry, blueberry, strawberry, or elderberry). Anthocyans may be up to 5000 mg/kg fresh weight in berries. The aglycones most commonly present in nature (anthocyanidins) are: pelargonidin, cyanidin, delphinidin, peonidin, petunidin, malvidin. Berries contain about 15 different anthocyans. Anthocyans are found in particularly high concentrations in fruits (berries) of plants of the genus Vaccinium, such as cranberry and blueberry.

The anthocyans present in the berries of the plants of the genus Vaccinium, (i.e. cranberry and blueberry), such as cyanidin, delphinidin, malvidin, petunidin and peonidin, are predominantly bound to a glycosidic residue and they are present in said berries, for example, such as cyanidin-3-arabinoside, cyanidin-3-galactoside, cyanidin-3-glucoside, delphinidin-3-arabinoside, delphinidin-3-galactoside, delphinidin-3-glucoside, malvidin-3-arabinoside, malvidin-3-galactoside, malvidin-3-glucoside, petunidin-3-arabinoside, petunidin-3-galactoside, petunidin-3-glucoside, peonidin-3-arabinoside, peonidin 3-galactoside, peonidin-3-glucoside.

Other ingredients which may be present in the extracts of the berries of the present invention are saccharides, organic acids and other polyphenols, such as flavonoids and tannins, as well as vitamins.

As concerns the polyphenol content of the extracts of berries of the present invention, there are differences between the selected berries. Specifically, the profile of the cranberry is distinguished by the richness of type A procyanidin; the predominant anthocyanidins are cyanidin and peonidin 3-O-monoglycosides; furthermore, cranberry also contains considerable amounts of phenolic acid and flavanols. On the contrary, blueberry is generally rich in anthocyanidins, in particular malvidin, B-type procyanidins and chlorogenic acid. The other extracts of berries (i.e. strawberry and elderberry) are characterised by a different composition; i.e. cyanidin monoglycosides, constituting about 10% elderberry, and ellagitannins and pelargonidin glycosides prevalent in strawberry.

Said at least one extract of at least one species of berries comprising or, alternatively, consisting of the polyphenolic fraction of said berries (extract of the invention) may be a single extract of a single species of berries or, alternatively, a single extract of 2 or 3 or 4 species of berries or, alternatively, 2 or 3 or 4 extracts, each extract being an extract of only one species of berries or, alternatively, of 2 or 3 or 4 species of berries. Preferably, the extract of the invention is only one extract of only one species of berries. Examples of berries that can be used in the context of the present invention to obtain said extract of the invention are reported hereinafter in the experimental part and in Table 1.

Said extract of at least one species of berries (for example, cranberry, blueberry, strawberry, or elderberry) comprised in the mixtures or compositions of the present invention comprises or, alternatively, consists of polyphenols (for example, proanthocyanidins (of type A and/or of type B) and/or anthocyanins and/or anthocyanidins) at a percentage by weight comprised in the range from 50% to 95% with respect to the total weight of the extract or dry extract (for example, 55%, 60%, 65%, 70%, 75%, 80%, 85%, 90%, 95%, 97%, or 98%); preferably from 70% to 97%; more preferably from 80% or 85% to 95%.

Anthocyanin levels in the extracts of the invention can be determined by means of an external calibration using standard substances.

Said at least one species of berries of said extract of the invention is selected from the group comprising or, alternatively, consisting of blueberry or European blueberry (Vaccinium cyanococcus or Vaccinium myrtillus or Vaccinium angustifolium), oxycoccus or cranberry (Vaccinium oxycoccos or Vaccinium macrocarpon), wild strawberry or strawberry (Fragaria vesca or Fragaria spp. or Fragaria ananassa), elderberry (Sambucus nigra), cowberry (Vaccinium vitis-idaea), blackberry (Rubus ulmifolius), red raspberry (Rubus idaeus), black raspberry (Rubus leucodermis or Rubus occidentalis), black currant (Ribes nigrum), red currant (Ribes rubrum), black mulberry (Morus nigra), red mulberry (Morus rubra), white mulberry (Morus alba), cornelian cherry (cornus mas), gooseberry (Ribes uva-crispa), barberry (berberis vulgaris), Amelanchier ovalis, Amelanchier canadensis, mahaleb cherry (Prunus mahaleb), sour cherries and black cherries (Prunus cerasus), strawberry tree (Arbutus unedo);

preferably blueberry or European blueberry (Vaccinium cyanococcus or Vaccinium myrtillus or Vaccinium angustifolium), oxycoccus or cranberry (Vaccinium oxycoccos or Vaccinium macrocarpon), wild strawberry or strawberry (Fragaria vesca or Fragaria spp. or Fragaria ananassa), elderberry (Sambucus nigra), and mixtures thereof;

more preferably blueberry or European blueberry (Vaccinium cyanococcus or Vaccinium myrtillus or Vaccinium angustifolium), and oxycoccus or cranberry (Vaccinium oxycoccos or Vaccinium macrocarpon).

The mixture (B) of the invention, of the composition (B), may comprise 1, 2, 3, 4, 5 or 6 bacterial strains selected from group (I) as defined in the present invention.

Advantageously, in said mixture (B) comprising 2, 3, 4, 5 or 6 bacterial strains selected from group (I.i), the bacterial strains are at a CFU ratio with respect to each other of about 1:1 or 1:1:1 or 1:1:1:1 or 1:1:1:1:1 or 1:1:1:1:1:1.

In an embodiment of the present invention, the composition (B) of the invention comprising the mixture (B) comprising or, alternatively, consisting of
at least one or a mixture of bacterial strains selected from group (I.i) comprising or, alternatively, consisting of: Lactobacillus paracasei DG® (CNCM I-1572), Lactobacillus paracasei LPC-S01™ (DSM 26760), Bifidobacterium bifidum MIMBb23SG (DSM 32708), Lactobacillus paracasei CF3 (DSM 32353), Lactobacillus rhamnosus GG (DSM 53103), Bifidobacterium animalis subsp. lactis Bb12 (DSM 15954), and mixtures thereof; and
at least one extract of at least one species of berries, preferably wherein said at least one species of berries is selected from the group comprising or, alternatively, consisting of: cranberry, blueberry, strawberry, elderberry and mixtures thereof, more preferably cranberry, or blueberry and mixtures thereof, comprising or, alternatively, consisting of the polyphenol fraction of said berries; and wherein, optionally, said composition (B) comprises at least one food grade or pharmacological additive and/or excipient.

In an embodiment of the composition (B) of the invention, the mixture (B) comprises or, alternatively, consists of a bacterial strain B. bifidum MIMBb23sg (DSM 32708) and of said at least one extract of at least one species of berries, preferably wherein said at least one species of berries is selected from the group comprising, a or alternatively, consisting of: cranberry, blueberry, strawberry, elderberry and mixtures thereof more preferably cranberry, or blueberry and mixtures thereof, comprising or, alternatively, consisting of the polyphenol fraction of said berries.

In an embodiment of the composition (B) of the invention, the mixture (B) comprises or, alternatively, consists of a bacterial strain L. paracasei LPC-S01™ (DSM 26760) and of said at least one extract of at least one species of berries, preferably wherein said at least one species of berries is selected from the group comprising, a or alternatively, consisting of: cranberry, blueberry, strawberry, elderberry and mixtures thereof, more preferably cranberry, or blueberry and mixtures thereof, comprises or, alternatively, consists of the polyphenol fraction of said berries.

In an embodiment of the composition (B) of the invention, the mixture (B) comprises or, alternatively, consists of: a bacterial strain B. bifidum MIMBb23sg DSM 32708 and of at least one bacterial strain selected from the group (I.ii) comprising or, alternatively, consisting of: L. paracasei DG® (CNCM I-1572), L. paracasei LPC-S01™ (DSM 26760), L. paracasei CF3 (DSM 32353), L. rhamnosus GG (DSM 53103), B. animalis subsp. lads Bb12 (DSM 15954); and of said at least one extract of at least one species of berries, preferably wherein said at least one species of berries is selected from the group comprising, a or alternatively, consisting of: cranberry, blueberry, strawberry, elderberry and mixtures thereof, more preferably cranberry, or blueberry and mixtures thereof, comprising or, alternatively, consisting of the polyphenol fraction of said berries.

In a preferred embodiment of the composition (B) of the invention, the mixture (B) comprises or, alternatively, consists of: a bacterial strain B. bifidum MIMBb23sg (DSM 32708) and a bacterial strain L. paracasei LPC-S01™ (DSM 26760) and of at least one extract of at least one species of berries, preferably wherein said at least one species of berries is selected from the group comprising, a or alternatively, consisting of: cranberry, blueberry, strawberry, elderberry and mixtures thereof, more preferably cranberry, or blueberry and mixtures thereof, comprising or, alternatively, consisting of the polyphenol fraction of said berries.

In an embodiment of the composition (B) of the invention, the mixture (B) comprises or, alternatively, consists of: a bacterial strain B. bifidum MIMBb23sg (DSM 32708) and a bacterial strain L. paracasei DG® (CNCM I-1572), and of said at least one extract of at least one species of berries, preferably wherein said at least one species of berries is selected from the group comprising, a or alternatively, consisting of: cranberry, blueberry, strawberry, elderberry and mixtures thereof more preferably cranberry, or blueberry and mixtures thereof, comprising or, alternatively, consisting of the polyphenol fraction of said berries.

In a preferred embodiment of the composition (B) of the invention, the mixture (B) comprises or, alternatively, consists of: a bacterial strain B. bifidum MIMBb23sg DSM 32708, a bacterial strain L. paracasei LPC-S01™ (DSM 26760) and of at least one bacterial strain selected from the group (I.iii) comprising or, alternatively, consisting of: L. paracasei DG® (CNCM I-1572), L. paracasei CF3 (DSM 32353), L. rhamnosus GG (DSM 53103), B. animalis subsp. lactis Bb12 (DSM 15954); and of said at least one extract of at least one species of berries, preferably wherein said at least one species of berries is selected from the group comprising, a or alternatively, consisting of: cranberry, blueberry, strawberry, elderberry and mixtures thereof, more preferably cranberry, or blueberry and mixtures thereof, comprising or, alternatively, consisting of the polyphenol fraction of said berries.

In a preferred embodiment of the composition (B) of the invention, the mixture (B) comprises or, alternatively, consists of: a bacterial strain B. bifidum MIMBb23sg DSM 32708 and a bacterial strain L. paracasei LPC-S01™ (DSM 26760) and a bacterial strain L. paracasei DG® (CNCM I-1572) and of said at least one extract of at least one species of berries, preferably wherein said at least one species of berries is selected from the group comprising, a or alternatively, consisting of: cranberry, blueberry, strawberry, elderberry and mixtures thereof, more preferably cranberry, or blueberry and mixtures thereof, comprising or, alternatively, consisting of the polyphenol fraction of said berries.

In the composition (B) of the invention, together with at least one or a mixture of bacterial strains defined in the present invention, preferably B. bifidum MIMBb23sg (DSM 32708) and/or L. paracasei LPC-S01™ (DSM 26760) and/or L. paracasei DG® (CNCM I-1572), more preferably B. bifidum MIMBb23sg (DSM 32708) and L. paracasei LPC-S01™ (DSM 26760), said at least one extract of at least one species of berries comprising or, alternatively, consisting of the polyphenol fraction (preferably an extract of cranberry, blueberry, strawberry, elderberry and/or mixtures thereof, more preferably an extract of cranberry, or blueberry and/or mixtures thereof), is present at a percentage by weight comprised in the range from 1% to 95% with respect to the total weight of the composition (for example, 5%, 10%, 15%, 20%, 25%, 30%, 35%, 40%, 45%, 50%, 55%, 60%, 65%, 70%, 75%, 80%, 85% or 90%), more preferably from 5% to 90%, even more preferably from 10% to 80%.

In a preferred embodiment of the composition (B) of the invention, the mixture (B) comprises or, alternatively, consists of: at least one or a mixture of bacterial strains selected from group (I) or (I.i), preferably B. bifidum MIMBb23sg (DSM 32708) and/or L. paracasei LPC-S01™ (DSM 26760) and/or L. paracasei DG® (CNCM I-1572), more preferably B. bifidum MIMBb23sg (DSM 32708) and L. paracasei LPC-S01™ (DSM 26760); and an extract of cranberry comprising or, alternatively, consisting of polyphenols (i.e. proanthocyanidins and/or anthocyanins and/or anthocyanidins and/or other polyphenols) at a percentage by weight comprised in the range from 70% to 99% with respect to the total weight of the extract (for example, 75%, 80%, 85%, 90%, 95%, 97%, or 98%); preferably from 80% to 97%; more preferably from 85% to 95%.

In a preferred embodiment of the composition (B) of the invention, the mixture (B) comprises or, alternatively, consists of: at least one or a mixture of bacterial strains selected from group (I) or (I.i), preferably B. bifidum MIMBb23sg (DSM 32708) and/or L. paracasei LPC-S01™ (DSM 26760) and/or L. paracasei DG® (CNCM I-1572), more preferably B. bifidum MIMBb23sg (DSM 32708) and L. paracasei LPC-S01™ (DSM 26760); and an extract of blueberry comprising or, alternatively, consisting of polyphenols at a percentage by weight comprised in the range from 70% to 95% with respect to the total weight of the extract (for example, 75%, 80%, 85%, 90%, 95%, 97%, or 98%); preferably from 80% to 97%; more preferably from 85% to 95%.

The amount, per daily dose of said composition (A) or (B), of said at least one or a mixture of bacterial strains comprised in said mixture (A) or (B) of the invention is the minimum amount sufficient to achieve temporary colonisation of the intestine, such as an amount of bacterial strain(s) comprised in the range from 10⁵ CFU/g to 10¹² CFU/g, preferably from 10⁷ CFU/g to 10¹¹ CFU/g, more preferably from 10⁸ CFU/g to 10¹⁰ CFU/g, for example 1×10⁹ CFU or 5×10⁹ CFU, with respect to the daily intake (CFU/g: colony-forming unit or gram of composition (A) or (B) of the invention). Said amounts of bacterial strain(s) may refer to amounts for each bacterial strain in said daily intake or to the total amount of bacterial strains comprised in said daily intake. Alternatively, said amounts of bacterial strain(s) may refer to amounts for each bacterial strain in dose units or to total amount of bacterial strains comprised in dosage units; a dose unit can be administered several times a day (for example 2 or 3 or 4 times a day).

In the context of the present invention, the bacterial strains can be or derive from: probiotic bacteria (live and viable), tyndalized bacteria, inactivated bacteria (for example by means of gamma irradiation or sonication), paraprobiotics, bacteria in the form of lysate or extracts (for example cell wall extract) or any derivative and/or component of bacteria, preferably, hexopolysaccharide, parietal fraction, metabolites or metabolic bioproducts generated by bacteria (postbiotics) and/or any other bacterial-derived product.

Preferably, the bacterial strains of the present invention are probiotic bacterial strains, such as “live and viable microorganisms which, when administered in adequate amounts, confer health benefits on the host” (FAO and WHO definition).

Besides the bacterial strains of the invention and, if present, besides said at least one extract of at least one species of berries, the composition (A) and composition (B) of the invention, optionally comprise said at least one pharmaceutical or food grade additive and/or excipient, i.e. a substance devoid of therapeutic activity suitable for pharmaceutical or food use. In the context of the present invention “additives and/or excipients acceptable for pharmaceutical or food use” comprise all auxiliary substances known to the man skilled in the art for the preparation of compositions in solid, semi-solid or liquid form, such as, for example, diluents, solvents (including water, glycerine, ethyl alcohol), solubilisers, acidifiers, thickeners, sweeteners, flavour enhancers, colouring agents, lubricants, surfactants, preservatives, pH stabilising buffers and mixtures thereof.

Besides the bacterial strains of the invention and, if present, besides said at least one extract of at least one species of berries the compositions (A) and (B) of the invention may advantageously further comprise at least one further component (component with inflammation or inflammation-related target activity) selected from the group comprising or, alternatively, consisting of: amino acids, vitamins of group A, B, C, D, E, K, magnesium, zinc and selenium organic and/or inorganic salts, immunostimulant substances, melatonin, valerian, passion flowers, lemon balm, hawthorn, chamomile, hops, antioxidants, anti-radical agents, prebiotic substances (for example, fructooligosaccharides (FOS), galactooligosaccharides (GOS), inulin, guar gum, glycosaminoglycans (for example, hyaluronic acid, chondroitin sulphate), collagen, substances acting on the serotoninergic pathway (e.g. cannabinoids), botanical extracts, enzymes and combinations thereof.

The compositions (A) and (B) of the invention, according to the various embodiments described in the present description, can be in solid form, such as tablet, chewable tablet, tablet to be dissolved in the mouth or mouth-soluble tablet, capsule, lozenge, granules, flakes or powder (granules or powder to be dissolved in a liquid or mouth-soluble granules), in semi-solid form, such as soft-gel, cream, or in liquid form, such solution, suspension, dispersion, emulsion or syrup.

The compositions (A) and (B) of the invention, according to the various embodiments described in the present description, can be formulated for oral (or gastroenteric), sublingual (or buccal), transmucosal, transdermal and/or topical use or administration, such as rectal, cutaneous, vaginal; they are preferably formulated for oral use.

The compositions (A) and (B) of the invention, according to the various embodiments described in the present description, may be a pharmaceutical composition (Live Biotherapeutic Products, LBP), a medical device composition, a dietary supplement or a food or a composition for a food for special medical purposes (FSMP) or novel food or probiotic products, and/or a cosmetic composition.

In the context of the present invention, the expression “medical device” is used in the meaning according to the Italian Legislative Decree no 46 dated 24 Feb. 1997 or according to the new Medical Device Regulation (EU) 2017/745 (MDR).

Forming a further object of the present invention are the compositions (A) and (B) of the invention, according to the various embodiments described in the present description, for use as medicament.

The compositions (A) and (B) of the invention may also be for use as medicament as adjuvants of further therapeutic approaches, preferably of a pharmacological, food or socio-behavioural type.

In an embodiment, the compositions (A) and (B) of the present invention, according to the various embodiments described in the present description, are for use as an immunomodulatory and/or immunostimulatory agent in a subject in need.

In the context of the present invention, the term “immunomodulatory and/or immunostimulatory agent” is used to indicate an agent and/or substance capable of varying the activity of the immune system, preferably capable of increasing and enhancing the activity of the immune system (for example, by modulating and/or stimulating the suitable pro-inflammatory and/or anti-inflammatory cytokines).

Advantageously, the composition (A) and the composition (B) of the present invention are capable of reducing the production of pro-inflammatory cytokines, preferably IL12 and/or TNF-α, and/or increasing the production of anti-inflammatory cytokines, preferably IL10. In particular, the composition (A) and the composition (B) of the present invention are capable of generating an IL12: IL10 ratio greater than 1 in the presence of inflammatory stimuli.

Based on the above description, the composition (A) and the composition (B) of the invention may be for use in a method for the preventive and/or curative treatment of diseases or symptoms and/or disorders caused by or related with/accompanied by an increase in pro-inflammatory cytokines and/or a reduction in anti-inflammatory cytokines, preferably diseases affecting: locomotor system (muscular and skeletal system), digestive system, urogenital system (urinary system and genital system), respiratory system, integumentary system, immune system and/or circulatory system.

In particular, the composition (A) and the composition (B) of the present invention have a valid application for the preventive and/or curative treatment of diseases related with, alterations of the immune system, in particular autoimmune diseases and allergies, immunodeficiency diseases, diseases affecting the skin, such as acne, and/or atopic dermatitis.

In an embodiment, the composition (A) and the composition (B) of the present invention, according to the various embodiments described in the present description, are for use as anti-inflammatory agent in a subject in need.

In an embodiment, the composition (A) and the composition (B) of the invention, according to the various embodiments described in the present description, are for use in a method for use in a method for preventive and/or curative treatment of inflammatory gastrointestinal diseases, disorders and/or symptoms in a subject in need, such as Helicobacter pylori, peptic or gastric ulcer, duodenal ulcer, chronic inflammatory bowel disease (IBD), such as Crohn's disease and ulcerative colitis, microscopic colitis, diverticular disease and diverticulitis.

In an embodiment, the composition (A) and the composition (B) of the invention, according to the various embodiments described in the present description, are for use in a method for the preventive and/or curative treatment of inflammatory musculoskeletal inflammatory diseases, rheumatological diseases, inflammatory articular and/or post-surgery inflammatory diseases, preferably for use in methods for the treatment of osteoarthritis, rheumatoid arthritis and ankylosing spondylitis, in particular osteoarthritis of the knee and osteoarthritis of the joints in general.

In an embodiment, the composition (A) and the composition (B) of the invention, according to the various embodiments described in the present description, are for use in a method for the preventive and/or curative treatment of functional gastrointestinal disorders (FGIDs), such as irritable bowel syndrome (IBS) (IBS with diarrhoea, IBS with constipation, IBS with alternating constipation and diarrhoea, unclassified IBS), dyspepsia, pyrosis, oesophagus, stomach and duodenum disorders, SIBO (small intestinal bacterial overgrowth), disorders with sub-inflammatory conditions, for example in the elderly, in the diverticular disease.

Forming an object of the present invention is a method for the anti-inflammatory or immunomodulatory and/or immunostimulatory treatment of diseases and/or disorders defined in the present invention by administering a therapeutically effective amount of the composition (A) or of the composition (B) of the invention according to the various embodiments described in the present description, to a subject.

In the context of the present invention, the expression “subjects” is used to indicate human subjects or animal subjects (e.g. pets, such as dogs or cats or other mammals). Preferably, the compositions of the invention are for use in treatment methods for human subjects.

For the sake of clarity, with the aim of achieving the object of the present invention, the components (or active components) of the mixture (A) or of the mixture (B) of the invention, such as bacterial strains and the extract of berries in the present invention, may also be administered separately (preferably within a time interval ranging from 30 minutes to 60 minutes) and in any order but, preferably, the various strains or the strains and the extract are administered to a subject simultaneously, even more preferably in a single composition so as to obtain a more rapid effect and for ease of administration. When the components (or active components) of the mixture (A) or (B) of the invention, such as the bacterial strains and the extract of berries in the present invention, are administered in a single composition, said single composition corresponds to the composition (A) or (B) of the present invention.

Forming an object of the present invention is a process (in short, extraction process of the invention) for the preparation of said extract of at least one species of berries comprising or, alternatively, consisting of the polyphenolic fraction of said berries (as defined in the context of the present invention) comprising the steps of:
step 1: extracting at least one species of berries, preferably berries in the form of powder (or dried berries), with water comprising the steps of:
   step 1.1: suspending at least one species of berries or a mixture of species of berries in water to disperse in water,
   step 1.2: mixing said dispersion of step 1.1 to obtain a mixture of step 1.2,
   optional step 1.3: sonicating said mixture of step 1.2 to obtain a sonicated mixture of step 1.3,
   step 1.4: centrifuging said mixture of step 1.2 or said sonicated mixture of step 1.3 to obtain a mixture comprising an aqueous supernatant called aqueous phase of step 1 and a solid residue of step 1; followed by
step 2 (for example after separating said aqueous phase and said solid residue of step 1): extracting the solid residue of step 1 with alcoholic solvent, preferably methanol, comprising the steps of:
   step 2.1: suspending the solid residue of step 1 in alcoholic solvent, preferably methanol, to obtain a dispersion in alcoholic solvent,
   step 2.2: mixing said dispersion of step 2.1 to obtain a mixture of step 2.2.,
   optional step 2.3: sonicating said mixture of step 2.2 to obtain a sonicated mixture of step 2.3,
   step 2.4: centrifuging said mixture of step 2.2 or said sonicated mixture of step 2.3 to obtain a mixture comprising an alcoholic supernatant called alcoholic phase of step 2 and a solid residue of step 2; followed by
step 3: loading the aqueous phase of step 1 onto a reversed-phase solid phase and extracting—by eluting with an acid aqueous solution (for example 0.01 M HCl)—to obtain an eluate of step 3 containing sugars and organic acids and a solid phase of step 3, such as the residual reversed phase solid phase after the extraction of step 3; the eluate of step 3 is discarded; followed by
step 4: extracting the solid phase of step 3 with an alcoholic solvent, preferably an acidic aqueous solution of methanol (e.g. methanol containing 0.1% HCl), to obtain an eluate of step 4 containing a polyphenol fraction and a solid phase of step 4, such as the residual reversed phase solid phase after the extraction of step 4; followed by
step 5: extracting the solid phase of step 4 with a ketone solvent (ketone), preferably acetone, to obtain an eluate of step 5 containing a polyphenol fraction, preferably proanthocyanidins and/or polyphenols contained in berry fibres; followed by
step 6: combining the eluate of step 4 and the eluate of step 5 and eliminating the solvent, for example by means of vacuum evaporation, to obtain the extract (for example dry extract) of at least one species of berries comprising a polyphenol fraction according to the present invention (extract of the invention).

Subsequently to step 6, the extraction process according to the invention may further comprise the step 7 of determining the polyphenol content of the extract of the invention (for example dry extract) by means of a quality/quantity analysis method, preferably by means of the Folin-Ciocalteu assay or any other suitable method known to the man skilled in the art.

Advantageously, the extractions of step 1 and step 2 are carried out in containers which shield the light, such as for example dark test tubes.

The step 1.1 of suspending at least one species of berries or a mixture of species of berries in water is carried out using the berries defined in the present invention, preferably cranberries and/or blueberries.

The mixing step (step 1.1 and 2.1) is preferably carried out with a vortexing instrument for a period of time comprised from 1 minute to 10 or 30 minutes, preferably from 1 minute to 5 minutes, at room temperature. In the present invention, the expression room temperature is used to indicate a temperature comprised in the range from 10 or 15° C. to 25° C., preferably 20° C.

The sonicating step (step 1.2 and 2.2) is preferably carried out for a period of time comprised from 5 minutes to 30 or 60 minutes, preferably from 10 minutes to 20 minutes, at room temperature.

The centrifuging step (step 1.3 and 2.3) is preferably carried out at 2000-4000 revolutions, preferably 3000 revolutions, for a period of time comprised from 1 minute to 30 or 60 minutes, preferably from 5 minutes to 15 minutes, at room temperature. The sonication of the mixture has the purpose of enhancing a greater disintegration of the berries (or berry powder) and allowing a greater extraction of the polyphenolic component present therein.

The step 3 of loading on a reversed-phase solid phase is preferably carried out using a reversed phase SPE cartridge (SPE: solid-phase extraction), for example an SPE Strata-X® Polymeric Reversed Phase cartridge which is a reversed phase functionalised polymeric absorbent which provides for strong retention of neutral, acidic or basic compounds under washing conditions with aggressive organic phases.

The step of eliminating the solvent (step 6) is preferably carried out by means of vacuum evaporation, for example by means of a rotavapor, at a temperature comprised in the range from 30° C. to 50° C. or 60° C., preferably 40° C.

The berries were extracted using the extraction process of the invention so as to eliminate the water-soluble fraction (mainly containing sugars and organic acids) and extracting and combining the methanol-soluble fraction (mainly containing polyphenols) and the acetone-soluble fraction (containing polyphenols, such as, for example, proanthocyanidins and anthocyanins and/or anthocyanidins, comprised in the berry fibres).

Forming an object of the present invention is said extract of at least one species of berries comprising or, alternatively, consisting of the polyphenolic fraction of said berries (extract of the invention) obtainable by means of the extraction process of the invention as defined above (steps 1-6 or steps 1-7). Said extract of at least one species of berries (for example, cranberry, blueberry, strawberry, or elderberry) comprises or, alternatively, consists of polyphenols at a percentage by weight comprised in the range from 50% to 95% with respect to the total weight of the extract (for example, 55%, 60%, 65%, 70%, 75%, 80%, 85%, 90%, 95%, 97%, or 98%); preferably from 70% to 97%; more preferably from 80% or 85% to 95%.

Forming an object of the present invention is a composition (B) (composition (B) of the invention) comprising a mixture (B) (mixture (B) of the invention) comprising or, alternatively, consisting of:
at least one or a mixture of bacterial strains selected from group (I) or (I.i) as defined in the present invention, and
said at least one extract of at least one species of berries comprising or, alternatively, consisting of the polyphenol fraction of said berries (extract of the invention) obtainable by means of the extraction process of the invention as defined above (steps 1-6 or steps 1-7); and wherein, optionally, said composition (B) comprises at least one food or pharmacological grade additive and/or excipient.

Unless otherwise specified, the expression composition or mixture or extract or other comprising a component at an amount “comprised in a range from x to y” is used to indicate that said component may be present in the composition or mixture or extract or other at all amounts present in said range, even if not specified, extremes of the range comprised.

The expression “therapeutically effective amount” refers to the amount of composition, mixture and/or bacterial strain that elicits the biological or medicinal response in a tissue, system, mammal, or human being that is sought and defined by an individual, researcher, veterinarian, physician, or other clinician or health worker.

In the context of the present invention the term “novel food” is used in its meaning according to the EU Regulation 2015/2283 dated 25 Nov. 2015.

A first set of embodiments (FRa no) of the present invention are reported below.

FRa1. A composition B comprising a mixture B comprising, or alternatively, consisting of:
at least one bacterial strain selected from the group comprising or, alternatively, consisting of
a bacterial strain identified as Bifidobacterium bifidum MIMBb23sg deposited at Deutsche Sammlung von Mikroorganismen und Zellkulturen GmbH (DSMZ) under deposit number DSM 32708,
a bacterial strain identified as Lactobacillus paracasei DG® deposited by SOFAR S.p.A. at the National Collection of Cultures of Microorganisms of the Pasteur Institute in Paris under access number CNCM I-1572.,
a bacterial strain identified as Lactobacillus paracasei LPC-S01™ deposited at Deutsche Sammlung von Mikroorganismen und Zellkulturen GmbH (DSMZ) under access number DSM 26760,
a bacterial strain identified as Lactobacillus paracasei CF3 deposited at Deutsche Sammlung von Mikroorganismen und Zellkulturen GmbH (DSMZ) under access number DSM 32353,
a bacterial strain identified as Lactobacillus rhamnosus GG deposited at Deutsche Sammlung von Mikroorganismen und Zellkulturen GmbH (DSMZ) under access number DSM 53103,
a bacterial strain identified as Bifidobacterium animalis subsp. lactis Bb12 deposited at Deutsche Sammlung von Mikroorganismen und Zellkulturen GmbH (DSMZ) under deposit number DSM 15954, and mixtures thereof; and
at least one extract of at least one species of berries comprising or, alternatively, consisting of a polyphenolic fraction of said berries; and wherein, optionally, said composition B comprises at least one food grade or pharmacological additive and/or excipient.

FRa2. The composition B according to FRa1, wherein said at least one bacterial strain comprises the bacterial strain Bifidobacterium bifidum MIMBb23sg DSM 32708 and furthermore at least one further bacterial strain selected from the group comprising or, alternatively, consisting of:
Lactobacillus paracasei DG® deposited by SOFAR S.p.A. at the National Collection of Cultures of Microorganisms of the Pasteur Institute in Paris under access number CNCM I-1572,
Lactobacillus paracasei LPC-S01™ deposited at Deutsche Sammlung von Mikroorganismen und Zellkulturen GmbH (DSMZ) under access number DSM 26760,
Lactobacillus paracasei CF3 deposited at Deutsche Sammlung von Mikroorganismen und Zellkulturen GmbH (DSMZ) under access number DSM 32353,
Lactobacillus rhamnosus GG deposited at Deutsche Sammlung von Mikroorganismen und Zellkulturen GmbH (DSMZ) under access number DSM 53103,
Bifidobacterium animalis subsp. Lactis Bb12 deposited at Deutsche Sammlung von Mikroorganismen und Zellkulturen GmbH (DSMZ) under deposit number DSM 15954, and mixtures thereof.

FRa3. The composition B according to FRa1 or FRa1, wherein said at least one bacterial strain comprises the bacterial strain Bifidobacterium bifidum MIMBb23sg DSM 32708 and furthermore at least one further bacterial strain selected from the group comprising or, alternatively, consisting of:
Lactobacillus paracasei DG® deposited by SOFAR S.p.A. at the National Collection of Cultures of Microorganisms of the Pasteur Institute in Paris under access number CNCM I-1572,
Lactobacillus paracasei LPC-S01™ deposited at Deutsche Sammlung von Mikroorganismen and Zellkulturen GmbH (DSMZ) under access number DSM 26760, and a mixture thereof.

FRa4. The composition B according to any one of the preceding FRas, wherein said at least one species of berries is selected from the group comprising or, alternatively, consisting of: blueberry or European blueberry (Vaccinium cyanococcus or Vaccinium myrtillus or Vaccinium angustifolium), oxycoccus or cranberry (Vaccinium oxycoccos or Vaccinium macrocarpon), wild strawberry or strawberry (Fragaria vesca or Fragaria spp. or Fragaria ananassa), elderberry (Sambucus nigra), cowberry (Vaccinium vitis-idaea), blackberry (Rubus ulmifolius), red raspberry (Rubus idaeus), black raspberry (Rubus leucodermis or Rubus occidentalis), black currant (Ribes nigrum), red currant (Ribes rubrum), black mulberry (Morus nigra), red mulberry (Morus rubra), white mulberry (Morus alba), cornelian cherry (cornus mas), gooseberry (Ribes uva-crispa), barberry (berberis vulgaris), Amelanchier ovalis, Amelanchier canadensis, mahaleb cherry (Prunus mahaleb), sour cherries or black cherries (Prunus cerasus), strawberry tree (Arbutus unedo); blueberry or European blueberry (Vaccinium cyanococcus or Vaccinium myrtillus or Vaccinium angustifolium), oxycoccus or cranberry (Vaccinium oxycoccos or Vaccinium macrocarpon), wild strawberry or strawberry (Fragaria vesca or Fragaria spp. or Fragaria ananassa), elderberry (Sambucus nigra), and mixtures thereof.

FRa5. The composition B according to any one of the preceding FRas, wherein said at least one species of berries is selected from the group comprising or, alternatively, consisting of: blueberry or European blueberry (Vaccinium cyanococcus or Vaccinium myrtillus or Vaccinium angustifolium), oxycoccus or cranberry (Vaccinium oxycoccos or Vaccinium macrocarpon), wild strawberry or strawberry (Fragaria vesca or Fragaria spp. or Fragaria ananassa), elderberry (Sambucus nigra), and mixtures thereof; preferably wherein said at least one species of berries is selected from the group comprising or, alternatively, consisting of: blueberry, cranberry, and a mixture thereof.

FRa6. The composition B according to any one of the preceding FRas, wherein said polyphenolic fraction of said extract of at least one species of berries comprises proanthocyanidins and/or anthocyanins and/or anthocyanidins.

FRa7. The composition B according to any one of the preceding FRas, wherein said at least one bacterial strain comprises or, alternatively, consists of Bifidobacterium bifidum MIMBb23sg DSM 32708 and Lactobacillus paracasei LPC-S01™ DSM 26760.

FRa8. The composition B according to any one of the preceding FRas, wherein said at least one bacterial strain comprises or, alternatively, consists of Bifidobacterium bifidum MIMBb23sg DSM 32708 and Lactobacillus paracasei DG® CNCM I-1572.

FRa9. The composition B according to any one of the preceding FRas, wherein said at least one bacterial strain comprises or, alternatively, consists of Bifidobacterium bifidum MIMBb23sg DSM 32708 and furthermore Lactobacillus paracasei LPC-S01™ DSM 26760 or Lactobacillus paracasei DG® CNCM I-1572, and wherein said at least one extract of at least one species of berries comprising or, alternatively, consisting of the polyphenol fraction of said berries is an extract of blueberry or European blueberry (Vaccinium cyanococcus or Vaccinium myrtillus or Vaccinium angustifolium) or, alternatively, of oxycoccus or cranberry (Vaccinium oxycoccos or Vaccinium macrocarpon) comprising or, alternatively, consisting of the polyphenol fraction of said berries.

FRa10. The composition B according to any one of the preceding FRas 1 to 9 for use as medicament.

FRa11. The composition B according to any one of FRas 1 to 9 for use as an immunomodulatory and/or immunostimulatory agent capable of reducing the production of proinflammatory cytokines and/or increasing the production of anti-inflammatory cytokines;

preferably, wherein said composition is for use as an immunomodulatory and/or immunostimulatory agent capable of reducing the production of IL12 and/or TNF-α cytokines and/or of increasing the production of IL10 cytokines.

FRa12. The composition B for use according to FRa10 or FRa11, wherein said composition is for use as an anti-inflammatory agent.

FRa13. The composition B for use according to FRa12, wherein said composition is for use in a method for preventive and/or curative treatment of inflammatory gastrointestinal diseases, disorders and/or symptoms selected from the group comprising or, alternatively, consisting of: Helicobacter pylori, peptic or gastric ulcer, duodenal ulcer, chronic inflammatory bowel diseases (IBD), Crohn's disease, ulcerative colitis, microscopic colitis, diverticular disease and diverticulitis.

FRa14. The composition B according to any one of FRas 1 to 9 for use in a method for the preventive and/or curative treatment of musculoskeletal inflammatory diseases, rheumatological diseases, inflammatory articular and/or post-surgery inflammatory diseases.

FRa15. The composition B for use according to FRa14, wherein said composition is for use in a method of preventive and/or curative treatment of osteoarthritis, rheumatoid arthritis and/or ankylosing spondylitis; preferably osteoarthritis of the joints and/or osteoarthritis of the knee.

A second set of embodiments (FRb no) of the present invention are reported below.

FRb1. A composition A comprising a mixture A comprising a mixture comprising or, alternatively, consisting of:
a bacterial strain identified as Bifidobacterium bifidum MIMBb23sg and deposited at Deutsche Sammlung von Mikroorganismen and Zellkulturen GmbH (DSMZ) under deposit number DSM 32708, and at least one bacterial strain selected from the group comprising or, alternatively, consisting of:
a bacterial strain identified as Lactobacillus paracasei DG® and deposited by SOFAR S.p.A. at the National Collection of Cultures of Microorganisms of the Pasteur Institute in Paris under access number CNCM I-1572,
a bacterial strain identified as Lactobacillus paracasei LPC-S01 and deposited at Deutsche Sammlung von Mikroorganismen und Zellkulturen GmbH (DSMZ) under access number DSM 26760,
a bacterial strain identified as Lactobacillus paracasei CF3 and deposited at Deutsche Sammlung von Mikroorganismen und Zellkulturen GmbH (DSMZ) under access number DSM 32353,
a bacterial strain identified as Lactobacillus rhamnosus GG and deposited at Deutsche Sammlung von Mikroorganismen und Zellkulturen GmbH (DSMZ) under access number DSM 53103,
a bacterial strain identified as Bifidobacterium animalis subsp. lactis Bb12 and deposited at Deutsche Sammlung von Mikroorganismen und Zellkulturen GmbH (DSMZ) under deposit number DSM 15954, and mixtures thereof;
and wherein, optionally, said composition comprises at least one food grade or pharmacological additive and/or excipient.

FRb2. The composition A according to FRb1, wherein the mixture A comprises or, alternatively, consists of: Bifidobacterium bifidum MIMBb23sg DSM 32708 and a bacterial strain and a bacterial strain Lactobacillus paracasei LPC-S01 DSM 26760.

FRb3. The composition A according to Frb1 or FRb2, wherein the mixture A comprises or, alternatively, consists of: a bacterial strain Bifidobacterium bifidum MIMBb23sg DSM 32708, a bacterial strain Lactobacillus paracasei LPC-S01 DSM 26760 and of at least one bacterial strain selected from the group comprising or, alternatively, consisting of: Lactobacillus paracasei DG® CNCM I-1572, Lactobacillus paracasei CF3 DSM 32353, Lactobacillus rhamnosus GG DSM 53103, Bifidobacterium animalis subsp. lactis Bb12 DSM 15954 and mixtures thereof.

FRb4. The composition A according to FRb1, wherein the mixture comprises or, alternatively, consists of: a bacterial strain Bifidobacterium bifidum MIMBb23sg DSM 32708 and a bacterial strain Lactobacillus paracasei DG® CNCM I-1572.

FRb5. The composition A according to FRb4, wherein the mixture comprises or, alternatively, consists of: a bacterial strain Bifidobacterium bifidum MIMBb23sg DSM 32708, a bacterial strain Lactobacillus paracasei DG® CNCM I-1572 and of at least one bacterial strain selected from the group comprising or, alternatively, consisting of: Lactobacillus paracasei LPC-S01 DSM 26760, Lactobacillus paracasei CF3 DSM 32353, Lactobacillus rhamnosus GG DSM 53103, Bifidobacterium animalis subsp. lactis Bb12 DSM 15954 and mixtures thereof.

FRb6. The composition A according to any one of the preceding FRbs, wherein the mixture comprises or, alternatively, consists of a bacterial strain Bifidobacterium bifidum MIMBb23sg DSM 32708 and a bacterial strain Lactobacillus paracasei LPC-S01 DSM 26760 and a bacterial strain Lactobacillus paracasei DG® CNCM I-1572.

FRb7. The composition A according to any one of FRbs 1 to 6 for use as medicament.

FRb8. The composition A according to any one of FRbs 1 to 6 for use as an immunomodulatory and/or immunostimulatory agent capable of reducing the production of proinflammatory cytokines and/or increasing the production of anti-inflammatory cytokines;

preferably, wherein said composition is for use as an immunomodulatory and/or immunostimulatory agent capable of reducing the production of IL12 and/or TNF-α cytokines and/or of increasing the production of IL10 cytokines.

FRb9. The composition A for use according to FRbs 7 or 8, wherein said composition for use is for use as an anti-inflammatory agent.

FRb10. The composition A for use according to FRb 9, wherein said composition is for use in a method for the preventive and/or curative treatment of inflammatory gastrointestinal diseases, disorders or symptoms selected from the group comprising or, alternatively, consisting of: Helicobacter pylori, peptic or gastric ulcer, duodenal ulcer, chronic inflammatory bowel disease (IBD), Crohn's disease, ulcerative colitis, microscopic colitis, diverticular disease and diverticulitis.

FRb11. The composition A according to any one of FRbs 1 to 6 for use in a method for the preventive and/or curative treatment of musculoskeletal inflammatory diseases, rheumatological diseases, inflammatory articular and/or post-surgery inflammatory diseases.

FRb12. The composition A for use according to FRb 11, wherein said composition is for use in a method for the preventive and/or curative treatment of osteoarthritis, rheumatoid arthritis and/or ankylosing spondylitis; preferably osteoarthritis of the joints and/or osteoarthritis of the knee.

Table 1 shows, by way of example, the anthocyanin/anthocyanidin content of an extract of dry blueberries (powder) and an extract of fresh blueberries. Abbreviations: Dp—Delphinidin, Cyclidine, Pt—Petunidin, Pn—Peonidin, My—Malvidin, Pg—Pelargonidin, gal—galactoside, glc—glucoside, ara—arabinoside.

TABLE 1

| | | Dry blueberries | |
|---|---|---|---|
| | | (powder) | Fresh blueberries |
| Peak no | Anthocyans | [g/Kg ± S) | [g/Kg ± S) |
| 1 | Dp-3-gal | 0.09 ± 0.01 | 12.95 ± 0.86 |
| 2 | Dp-pentose-hexose | n.q. | — |
| 3 | Dp-3-glc | 0.45 ± 0.05 | 9.11 ± 0.43 |
| 4 | Cy-3-gal | 0.04 ± 0.01 | 8.69 ± 0.31 |
| 5 | Dp-3-ara | 0.18 ± 0.02 | 6.73 ± 0.39 |
| 6 | Cy-3-glc | 0.34 ± 0.03 | 12.92 ± 0.27 |
| 7 | Pt-3-gal | 0.07 ± 0.01 | 5.44 ± 0.39 |
| 8 | Cy-3-ara | 0.11 ± 0.01 | 6.00 ± 0.27 |
| 9 | Pt-3-glc | 0.45 ± 0.05 | 7.50 ± 0.87 |
| 10 | Pn-3-gal | n.q. | n.q. |
| 11 | Pt-3-ara | 0.21 ± 0.02 | 5.32 ± 0.42 |
| 12 | Pn-3-glc | 0.08 ± 0.01 | 7.75 ± 0.29 |
| 13 | Mv-3-gal | 0.11 ± 0.01 | 6.75 ± 1.22 |
| 14 | Mv-3-glc | 0.84 ± 0.10 | 6.35 ± 0.36 |
| 15 | Mv-3-ara | 0.64 ± 0.13 | 5.80 ± 0.59 |
| 16 | Pn-pent | n.q. | — |
| 17 | Mv-pent | n.q. | — |

n.q. = not quantified

### EXPERIMENTAL PART

The immunomodulatory capacity of compositions (A) and (B) of the invention was tested using a model of dendritic cells isolated from mouse bone marrow, i.e. Bone Marrow-derived Denditic Cells (in short BMDCs).

Materials

(I) Bacterial Strains:
L. paracasei DG® (CNCM I-1572), in short DG;
L. paracasei LPC-S01™ (DSM 26760), in short LPC-S01™;
L. paracasei CF3 (DSM 32353), in short CF3;
L. rhamnosus GG (DSM 53103), in short GG;
B. bifidum MIMBb23sg (DSM 32708), in short 23SG;
B. animalis subsp. Lactis Bb12 (DSM 15954), in short Bb12;

as defined in the present invention;

(II) Berries as starting material of the extraction process according to the invention:
“Wild blueberry powder, 3% polyphenols” (in short, 3% PP): produced by Naturex, product code OK705055, botanical species Vaccinium myrtillus or Vaccinium angustifolium. Qualitative analysis (by means of HPLC): polyphenol content>3% (from 3% to 5% or 10% or 20% or 30% or 40% or 50%) (area/HPLC area under the curve % or weight/weight %), loss on drying<5.00%, particle size: >95% by means of 30-mesh sieve (600 μm) and >95% by means of 100-mesh sieve (150 μm), bulk density 0.30-0.60 g/ml;
“Wild blueberry powder, 50% fibres” (in short, 50% FB): produced by Naturex, product code 0K705001, botanical species Vaccinium myrtillus or Vaccinium angustifolium. Qualitative analysis (by means of HPLC): polyphenol content>3% (from 3% to 4% or 5% or 6% or 8% or 10%, weight/weight %, fibre content>50%, loss on drying<5.00%, particle size: >60% by means of 60-mesh sieve (250 μm), bulk density 0.30-0.60 g/ml;
“Strawberry powder 100% fruit”: produced by Naturex, product code ON200003, botanical species Fragaria spp. Qualitative analysis (by means of TLC): loss on drying<3.00%, particle size: 100% through 1.4 mm;
“Cranberry 1% proanthocyanidins” (in short, 1% PA): produced by Naturex, product code CRANBERRY PE 1% PROANTHOCYANIDINS (Ref. EH711552), powder, botanical species Vaccinium macrocarpon (Ainton). Qualitative analysis: proanthocyanidin content (as cyanidin chloride, Ph Eur method 1200)>1% evaluated by means of HPLC method (CQ-MO-467) (value 1.92%), particle size: >90% by means of 300-mesh sieve (Sieve (CQ-MO-018), loss on drying<6.00% (IR balance (CQ-MO-018) (value 1.06%), (tap density) 0.4-0.8 g/ml (densimeter, CQ-MO-257), pH (solution 1%) 3-6 (pH meter CQ-MO-123);
“Cranberry 1% proanthocyanidins” (in short, 1% PA): produced by Naturex, product code NUTRICRAN® 90S-06155 (Ref. EK036155), powder, botanical species Cranberry red (Ainton). Qualitative analysis: proanthocyanidin content (PACs)>1.0% (method CQ-MO-232 subtracted from CQ-MO-203) (value 1.95% weight/weight), particle size: 100% by means of 30-mesh sieve and >95% by means of 100-mesh sieve (Screen analysis (LA-03-002-00), moisture<5.00% (IR balance (CQ-M0-018), bulk density 0.5-0.6 g/ml and tap density 0.6-0.8 g/ml (densimeter, CQ-MO-257), pH (solution 1%) 3-6 (pH meter (CQ-MO-123);
“Cranberry 1.5% proanthocyanidins”: produced by Naturex, product code PACRAN® EU-SP_06104 (Ref. GK006104), powder, botanical species Vaccinium macrocarpon (Ainton), proanthocyanidin content>1.5% evaluated by means of HPLC method (CQ-MO-583 subtracted from CQ-MO-582) (value 2.97%) (area/HPLC area under the curve % or weight/weight %), loss on drying<6.00% (IR balance (CQ-MO-018), tap density 0.5-0.7 g/ml (densimeter, CQ-MO-257), particle size: 100% by means of 80-mesh sieve, pH (1% solution) 2.6-3.9 (pH meter);
“Cranberry 15% proanthocyanidins” (in short, 15% PA): produced by Nutra, product code URO-std-Pur, powder, botanical species Vaccinium macrocarpon (Ainton), proanthocyanidin content 15.9% (BL-DMAC) (weight/weight %), loss on drying 3.3%, particle size: 100% by means of 80-mesh sieve;
“Elderberry dry fruit spray”: produced by Iprona, product code 70120053, powder, botanical species Sambucus nigra (L.), anthocyanin content expressed as cya-3-glu (spectrum in buffer pH 1.0) 88.5 g/Kg, polyphenol content expressed as catechin (Folin Ciocalteu) 109.0 g/Kg.

Polyphenol content in mg on 1 g of powder:
blueberry 3% PP: 74 mg/g;
blueberry 50% fibre: 51 mg/g;
strawberries: 115.5 mg/g;
cranberry 1% PA: 31.7 mg/g;
cranberry 15% PA: 158 mg/g;
elderberry: 122 mg/g.

Methods

(III) Bacterial Strains, Preparation and Growth Conditions:

All Lactobacillus strains used for this trial were cultured in De Man-Rogosa-Sharpe (MRS) broth (Difco Laboratories Inc., Detroit, Mich., USA). Bifidobacterium strains were cultured in MRS supplemented with 0.05% L-cysteine hydrochloride (Sigma-Aldrich) (cMRS). The bacteria were inoculated from frozen glycerol strains and sub-cultured twice in MRS or cMRS using a 1:100 inoculum; the Lactobacillus strains were incubated at 37° C., while the bifidobacteria were incubated at 37° C. under anaerobic conditions (naerocult A System; Merck, Darmstadt, Germany). Bacterial cells from an overnight culture were harvested and washed twice using sterile PBS (for bifidobacterial strains using prereduced cPBS). Thereafter, the total count using Neubauer Improved counting chamber was compared with the number of viable cells of bacterial suspensions conducted using an Accuri C6 flow cytometer (BD Biosciences, Milan, Italy) with staining of the propidium iodide cells. Based on the vital count, each bacterial strain was resuspended at a known concentration in prereduced cPBS added with sterile glycerol (1:6 v/v) and stored at −80° C. in aliquots. The viability of bacterial cells was controlled by diluting and plating—on MRS or cMRS agar—an aliquot for each strain after one week of storage at −80° C.

(IV) Extracting the Polyphenol Fraction from Berries

The berry extraction method was carried out following the method described by Wrolstad (Wrolstad et al, Handbook of analytical chemistry: pigments, colorants, flavour, texture and bioactive food components, vol 2. Wiley, New Jersey, pp 473-475, 2005) with some modifications, as described in the extraction method of the present invention.

In particular, 500 milligrams (mg) of berries in powder form, such as blueberry, cranberry, strawberry or elderberry, were dispersed in 40 ml of deionised water (step 1) (dark test tube to protect from light), mixed by vortexing for 3 minutes at a temperature of 20° C. Then, the aqueous dispersion of berries was sonicated for 15 minutes at a temperature of 20° C. and, subsequently, the sonicated mixture was centrifuged at 3000 rpm for 10 minutes at a temperature of 20° C. providing a mixture comprising an aqueous supernatant (aqueous phase of step 1) and a solid residue (solid residue of step 1). The aqueous supernatant was recovered (aqueous phase of step 1) and the solid residue (solid residue of step 1) was extracted a second time using 10 ml of methanol (step 2; extraction method similar to that described for step 1) and providing an alcoholic supernatant (alcohol phase of step 2) and a solid residue (solid residue of step 1).

The separation of the components contained in the aqueous supernatant of step 1 and in the alcoholic supernatant of step 2 was carried out through extraction using a solid phase extraction (SPE) cartridge. In detail, a volume of 6 ml of aqueous supernatant of step 1 was loaded onto an SPE cartridge (Strata-X®, Polymeric Reversed Phase, 200 mg/6 mL) and the water-soluble phase containing sugars and organic acids was eluted using 0.01 N HCl (5 mL) (step 3) as mobile phase; the eluate of step 3 containing sugars and organic acids was discarded. Subsequently, the alcoholic supernatant of step 2 (10 ml) was loaded onto said SPE cartridge and the polyphenol fraction was eluted using methanol containing 0.1% HCl (5 ml) (step 4) as mobile phase. Lastly, the SPE cartridge was eluted using acetone (step 5) to extract and recover the proanthocyanidins and the polyphenols present in the berry fibres in the eluted fraction.

The fraction eluted according to step 4 and the fraction eluted using acetone according to step 5 were combined and evaporated by means of rotavapor at a temperature of 40° C. to obtain the extract of berries comprising a polyphenol fraction according to the present invention (in short, the extract of the invention).

The obtained extract of the invention was dissolved in methanol acidified with HCl (0.05 mm) and the obtained solution was analysed for the total polyphenol content by means of the Folin-Ciocalteu assay.

The percentage by weight (with respect to the total weight of the extract) of the total polyphenols extracted from the aforementioned berries by means of the extraction method of the invention was higher than 90% (from 90% to 91% or 92% or 93% or 94% or 95% or 96% or 97% or 98% or 99%; the analysis was carried out by means of the Folin Ciocalteu method.

Polyphenol content in mg on 1 ml of extract (the results were expressed as gallic acid equivalents (GAE) using a calibration curve obtained using the gallic acid standard):
blueberry 3% PP: 36.93 mg/ml;
blueberry 50% fibre: 25.54 mg/ml;
strawberries: 57.80 mg/ml;
cranberry 1% PA: 15.85 mg/ml;
cranberry 15% PA: 78.99 mg/ml;
elderberry: 60.59 mg/ml.

The extracts obtained using the different species of berries, such as blueberry, cranberry, strawberry and elderberry, were stored at −20° C. up to the time of use in immunomodulation experiments with the dendritic cells.

On the day of the in vitro experiment with bone marrow derived dendritic cells (BMDCs), the extract was dissolved in RPMI medium (dendritic cell growth medium) at the final use concentration of 50 μg/ml, based on the quantification of the total polyphenol content carried out using the Folin-Ciocalteu assay reported above.

(V) Folin-Ciocalteu Assay_Analysis of the Polyphenol Fraction of the Extract of the Invention.

The analysis was carried out using a liquid chromatographic system which consisted in an Alliance 2695 model (Water, Milford, Mass., USA) equipped with a model 2998 (waters) photodiode array detector. The separation was carried out through a C 18 Kinetex column (150×4.6 mm, 2.6 μm, Fenomenex) at 45° C. with a minimum flow rate of 1.7 ml/min. The eluents were (A) 1% H₃PO₄ and (B) acetonitrile/water (35:65, v/v). The elution gradient was linear as indicated: 0-15 min, 14% B; 15-25 minutes, from 14 to 20% B; 25-35 minutes, from 20 to 32% B; 35-45 minutes, from 32 to 50% B; 45-48 min, from 50 to 90% B; 90% for 3 minutes. The chromatographic data were acquired from 200 to 700 nm and integrated at 520 nm (ACN) and 320 nm (Phe). Calibration curves from 2 to 50 μg/ml were obtained for Cy-, Dp-, Pt-, Pe- and Mv-3-O-glc, Cy- and Pt-3-O-gal and Pt-3-O-ara and chlorogenic acid. The working solution was diluted from the stock solution using methanol acidified with 0.1% TFA. Each test was performed in duplicate. The identification of the individual ACNs was confirmed by the LC coupled to electrospray ionization-mass spectrometry (ESI-MS) as previously described by Del Bo′ et al. (Del Bo′ C ET AL, J Agric Food Chem. 2010 Feb. 24; 58(4):2491-7). In short, the mass spectrometer operates in positive full scan mode in the range 200 Da-800 Da. The capillary voltage was set to 3.5 kV, the cone voltage at 20 V, the original temperature at 130° C. and the desolvation temperature at 350° C. The data were acquired from the Masslinx 4.0 software (Micromass, Beverly, Mass., USA).

(VI) Generation of Bone Marrow-Derived Dendritic Cells (BMDCs)

BMDCs (bone marrow-derived dendritic cells) were obtained by isolating monocytes collected from tibia and femur bone marrow from 6-12-week-old C57BL/6 mice. After being removed, tibia and femur were treated for 2 minutes with EtOH and subsequently for 2 minutes with sterile PBS. Monocytes were obtained by washing tibia and femur with syringes containing sterile PBS.

The recovered cells were centrifuged for 10 minutes at 1200 rpm at 4° C. The supernatant was removed and the cellular pellet was washed again with PBS and centrifuged under the same conditions. The cellular pellet was subsequently resuspended in 10 ml of RPMI 1640 medium (RPMI 1640: Rosewell Park Memorial Institute 1640 Medium) containing L-glutamine (4 mm), thermally inactivated FBS (foetal calf serum) 10% v/v, penicillin (100 U/ml), streptomycin (100 mg/ml), 50 mm 2-mercaptoethanol, with addition of GMCSF (granulocyte macrophage colony-stimulating factor) at the final concentration of 15 ng/ml.

The cells were counted using a counting chamber (Fuchs-Rosenthal) and brought to a concentration of 3.5×10⁵ cells/ml, aliquoted in Petri dishes (each containing 10 ml of cell suspension) and placed to differentiate in the presence of Granulocyte Macrophage Colony-stimulating Factor (GMCSF) at an amount of 15 ng/ml for 87 days. The medium with GMCSF was replaced with fresh medium on the third and fifth day of differentiation. On day 8 the cells were recovered from each Petri dish, centrifuged and resuspended at the concentration of 2×10⁶ cells/ml in complete RPMI medium without GMCSF.

(VII) Stimulation of Dendritic Cells with Compositions (A) or (B) of the Invention

The dendritic cells (1×10⁶ BMDCs) were placed at contact with:

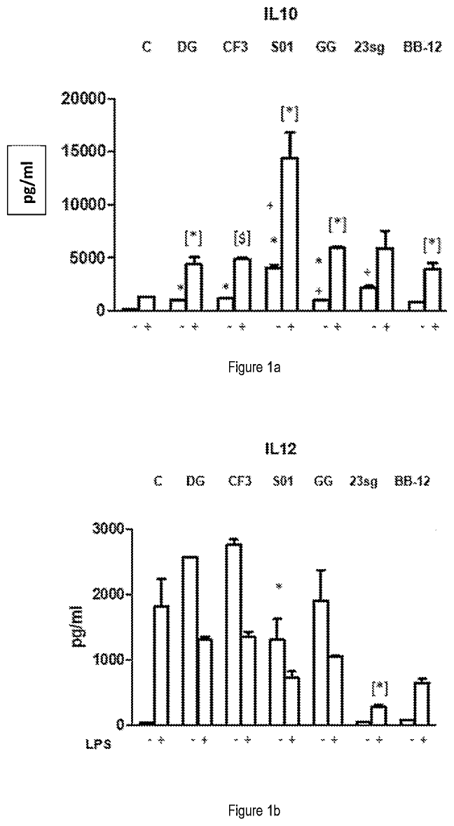

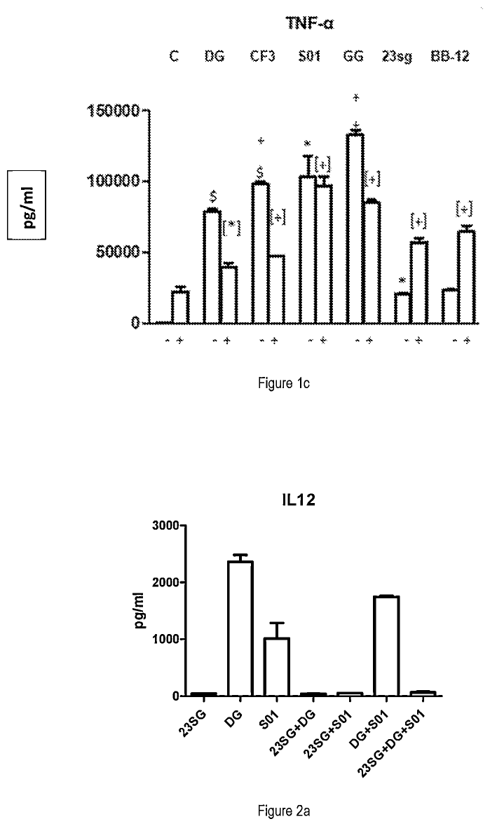

(a) individual bacterial strains listed in paragraph (I) (FIG. 1a, 1b, 1c);

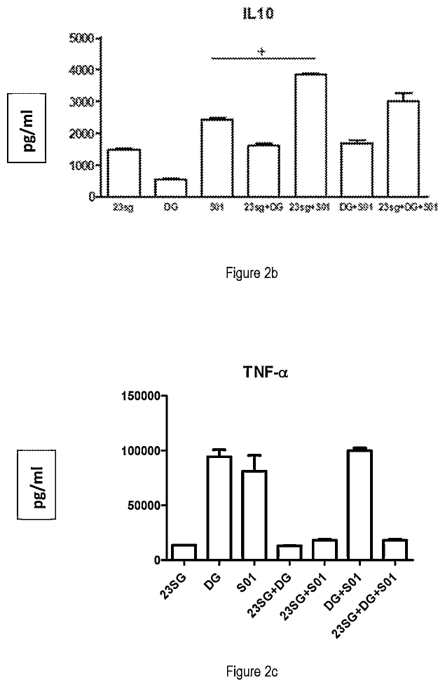

(b) mixtures of at least 2 bacterial strains listed in paragraph (I) (compositions (A) according to the invention: DG+LPC-S01™, DG+MIMBb23sg, LPC-S01™+MIMBb23sg, MOI, total final MOI of 5) (FIG. 2a, 2b, 2c);

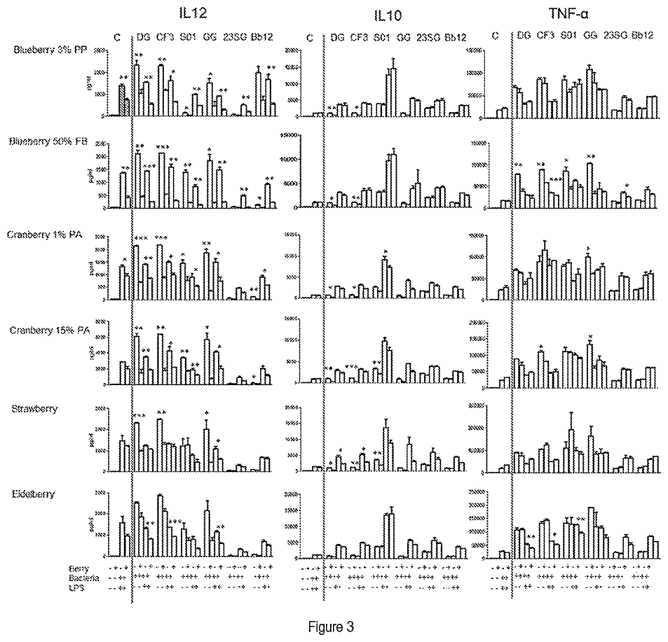

(c) mixtures of a bacterial strain selected from the strains listed in paragraph (I) and an extract of a species of berries selected from the species of berries listed in paragraph (II), wherein the extraction is according to the extraction method of the invention to obtain extracts comprising the polyphenol fraction (compositions (B) according to the invention) (FIG. 3);

(d) mixtures of at least 2 bacterial strains selected from the strains listed in paragraph (I) and an extract of a species of berries selected from the species of berries listed in paragraph (II), wherein the extraction is according to the extraction process of the invention to obtain extracts comprising the polyphenol fraction (compositions (B) according to the invention) (FIG. 4).

The cells were stimulated both in the absence and in the presence of a proinflammatory stimulus obtained using lipopolysaccharide (LPS) from Escherichia coli, used at an amount of 1 μg/ml.

The stimulation of BMDCs with (a), (b), (c), (d) as defined above and LPS was carried out by means of incubation at 37° C. and 5% CO₂ for 20 hours. Subsequently, the supernatant was collected without removing the cells present at the bottom of the well and used to evaluate the production of IL12, TNF-α and IL10 pro- and anti-inflammatory cytokines using the ELISA immunoenzymatic assay.

In particular, the following cytokines were evaluated:
TNF-α (tumour necrosis factor-alpha) pro-inflammatory cytokine;
IL-10 (interleukin-10) anti-inflammatory cytokine; and
IL-12 (interleukin-12), the stimulatory cytokine responsible for the activation of adaptive immunity.

Each experiment included a control condition (i.e. BMDCs stimulated with RPMI medium only), a control in the presence of MetOH-HCl (corresponding to the same amount present in each tested extract, and always lower than 0.1% v/v with respect to the volume of cell suspension in each well) and LPS+MetOH-HCl.

All strains were tested both in the absence and in the presence of MetOH-HCl, with and without LPS.

The assay was carried out in 96-well multiwell plates whose bottom was pre-treated and coated with 50 μl of the capture antibody resuspended in PBS specific for each cytokine of interest (treatment lasted overnight at 4° C.). Then the plates were washed with a buffer containing 8 g/l NaCl, 1.44 g/l Na₂HPO₄, 0.24 g/l KH₂PO₄, 0.05% Tween20, pH 7.4 and blocked with 250 μl of PBS+1% Bovine Serum Albumin (BSA) solution for 1 hour at room temperature. Then, the plates were washed and the supernatants corresponding to the various samples tested were added. The supernatants were diluted in a solution consisting of PBS+1% BSA, at a 1:2 ratio for IL12, 1:10 for IL10 and 1:100 for TNF-α, final volume in the wells 50 μl. Eight 1:2 dilutions of the standard solution of each cytokine were added in technical duplicate in each plate for the construction of the calibration line required for the quantification of proteins in supernatants. After 2 hours incubation at room temperature, the plates were washed and treated with 50 μl of secondary antibody conjugated with biotin resuspended at room temperature for 2 hours. After washing the plates, the conjugated treptavidine-horse radish peroxidase enzyme, diluted in a detection solution was added in a final volume of 50 μl per well. The plates were incubated for 20 min. After washing, the plates were further washed with distilled water and incubated with tetramethylbenzidine (peroxidase activity detector, TMB) resuspended in a specific solution and allowed to incubate for another 20 minutes at room temperature for the development of the colorimetric reaction depending on the enzymatic activity. Subsequently, 100 μl of a H₃PO₄ 2 M solution were added to each well to block the reaction and the absorbance reading at 450/630 nm was carried out using a spectrophotometer. The colour intensity of each sample was compared with the standard curve, giving a quantitative result in terms of optical density (OD) and concentration based on the dilutions used.

In the preliminary step, experiments were carried out to evaluate the suitable amount of extracts to be used in contact with BMDCs, with the aim of excluding a potential effect on dendritic cells by the solvent (MetOH-0.05 mM HCl) used to obtain the berry extracts comprising the polyphenol fraction. Following these preliminary tests, it was decided to work with an amount of 50 μg/ml of berry extract content comprising the polyphenol fraction, corresponding to an amount of MetOH-HCl in contact with the dendritic cells lower than 0.1% (v/v).

As concerns bacterial strains, a preliminary evaluation was carried out to determine the amount to be used at contact with BMDCs.

Based on these tests, it was decided to test the bacterial strains at a MOI (multiplicity of infection) with respect to the number of BMDCs equal to 5, corresponding to a total amount of bacterial cells of 5×10⁶.

When used in the absence of extracts, the bacterial strains were added with the amount of MetOH-HCl corresponding to the one present in 50 μg/ml of each tested extract, so as to be able to attribute the potential greater/synergistic effect to the presence of bioactive components in the berries and not to the presence of MetOH-HCl.

Also in the co-incubation experiments with LPS a control was always included in the presence of MetOH-HCl.

All the experiments were carried out in technical duplicate and in biological duplicate.

(VIII) Statistical Analysis

Statistical calculations were carried out using the GraphPad Prism 5 software program. The meaning of the results was analysed by means of unpaired heteroscedastic Student's t-tests with two-tail distribution. Differences of P<0.05 were considered significant.

Results

(IX) Evaluation of the Compositions (A) of the Invention
II B. bifidum MIMBb23sg (23SG), when combined with L. paracasei DG® (23SG-FDG) or with L. paracasei LPC-S01™ (23SG-FLPC-S01™), reduces the stimulatory response thereof, as concerns both IL12 and TNF-α.
Furthermore, the combination of B. bifidum MIMBb23sg with L. paracasei LPC-S01™ (23SG+LPC-S01™) induces a higher production of IL10 with respect to the bacterial strains alone (synergistic effect).

Thus, the combination of B. bifidum MIMBb23sg and L. paracasei LPC-S01™ (235G+LPC-S01™) (composition (A) according to the invention) has an IL10:IL12 ratio much higher than 1 and a potential anti-inflammatory effect (anti-inflammatory immunostimulatory effect).

(X) Evaluation of the Composition (B) of the Invention
All the berry extracts according to the invention show a capacity to modulate the immune responses induced by the different bacterial strains tested individually (FIG. 3).

Alone, the berry extracts were not capable of inducing neither IL12 nor IL10.

In particular, the blueberry extracts (3% PP and 50% FB) and the cranberry extracts (1% PA and 15% PA) are both effective in reducing the production of IL12, at baseline and in the presence of LPS (FIG. 3).

Furthermore, the 50% FB blueberry extract is the most effective in reducing IL12 and TNF-α (pro-inflammatory cytokines) for all bacterial strains tested (FIG. 3).
The combination of berry extracts comprising the polyphenol fraction according to the invention (3% PP and 50% FB blueberry, 1% PA and 15% PA cranberry) with the best combination of bacterial strains, such as B. bifidum MIMBb23sg and L. paracasei LPC-S01™ (23SG+LPC-S01™), contributes to further inhibiting the production of IL-12 (pro-inflammatory cytokine), even in the presence of proinflammatory stimulus with LPS (FIGS. 4 and 4a).

Furthermore, the combination of 50% FB blueberry extract according to the invention or 1% PA cranberry extract with the combination of bacterial strains B. bifidum MIMBb23sg and L. paracasei LPC-S01™ (23SG+LPC-S01™) contributes to further inhibiting the production of TNF-α (pro-inflammatory cytokine), even in the presence of the proinflammatory stimulus with LPS (FIGS. 4 and 4a).

### CONCLUSIONS

The compositions according to the invention comprising one or more bacterial strains (i.e. 23SG+LPC-S01™) and the blueberry or cranberry extracts comprising the polyphenol fraction have a potential anti-inflammatory effect (anti-inflammatory immunostimulatory effect).

1. A composition comprising a mixture comprising:
at least one bacterial strain selected from the group consisting of
a bacterial strain identified as Bifidobacterium bifidum MIMBb23sg deposited at Deutsche Sammlung von Mikroorganismen und Zellkulturen GmbH (DSMZ) under deposit number DSM 32708,
a bacterial strain identified as Lactobacillus paracasei DG® deposited at the National Collection of Cultures of Microorganisms of the Pasteur Institute in Paris under access number CNCM I-1572 by SOFAR S.p.A.,
a bacterial strain identified as Lactobacillus paracasei LPC-S01™ deposited at Deutsche Sammlung von Mikroorganismen und Zellkulturen GmbH (DSMZ) under access number DSM 26760,
a bacterial strain identified as Lactobacillus paracasei CF3 deposited at Deutsche Sammlung von Mikroorganismen und Zellkulturen GmbH (DSMZ) under access number DSM 32353,
a bacterial strain identified as Lactobacillus rhamnosus GG deposited at Deutsche Sammlung von Mikroorganismen und Zellkulturen GmbH (DSMZ) under access number DSM 53103,
a bacterial strain identified as Bifidobacterium animalis subsp. lactis Bb12 deposited at Deutsche Sammlung von Mikroorganismen und Zellkulturen GmbH (DSMZ) under deposit number DSM 15954, and mixtures thereof; and
at least one extract of at least one species of berries comprising a polyphenol fraction of said berries;
and wherein, optionally, said composition comprises at least one food or pharmacological grade additive and/or excipient.

2. The composition according to claim 1, wherein said at least one bacterial strain comprises the bacterial strain Bifidobacterium bifidum MIMBb23sg DSM 32708 and at least one bacterial strain selected from the group consisting of:
Lactobacillus paracasei DG® deposited by SOFAR S.p.A. at the National Collection of Cultures of Microorganisms of the Pasteur Institute in Paris under access number CNCM I-1572,
Lactobacillus paracasei LPC-S01™ deposited at Deutsche Sammlung von Mikroorganismen und Zellkulturen GmbH (DSMZ) under access number DSM 26760,
Lactobacillus paracasei CF3 deposited at Deutsche Sammlung von Mikroorganismen und Zellkulturen GmbH (DSMZ) under access number DSM 32353,
Lactobacillus rhamnosus GG deposited at Deutsche Sammlung von Mikroorganismen und Zellkulturen GmbH (DSMZ) under access number DSM 53103,
Bifidobacterium animalis subsp. lactis Bb12 deposited at Deutsche Sammlung von Mikroorganismen und Zellkulturen GmbH (DSMZ) under deposit number DSM 15954,
and mixtures thereof.

3. The composition according to claim 1, wherein said at least one bacterial strain comprises the bacterial strain Bifidobacterium bifidum MIMBb23sg DSM 32708 and at least one bacterial strain selected from the group consisting of:
Lactobacillus paracasei DG® deposited by SOFAR S.p.A. at the National Collection of Cultures of Microorganisms of the Pasteur Institute in Paris under access number CNCM I-1572,
Lactobacillus paracasei LPC-S01™ deposited at Deutsche Sammlung von Mikroorganismen und Zellkulturen GmbH (DSMZ) under access number DSM 26760, and a mixture thereof.

4. The composition according to claim 1, wherein said at least one species of berries is selected from the group consisting of: blueberry or European blueberry (Vaccinium cyanococcus or Vaccinium myrtillus or Vaccinium angustifolium), oxycoccus or cranberry (Vaccinium oxycoccos or Vaccinium macrocarpon), wild strawberry or strawberry (Fragaria vesca or Fragaria spp. or Fragaria ananassa), elderberry (Sambucus nigra), cowberry (Vaccinium vitis-idaea), blackberry (Rubus ulmifolius), red raspberry (Rubus idaeus), black raspberry (Rubus leucodermis or Rubus occidentalis), black currant (Ribes nigrum), red currant (Ribes rubrum), black mulberry (Morus nigra), red mulberry (Morus rubra), white mulberry (Morus alba), cornelian cherry (cornus mas), gooseberry (Ribes uva-crispa), barberry (berberis vulgaris), Amelanchier ovalis, Amelanchier canadensis, marasca cherry (Prunus mahaleb), sour cherries and black cherries (Prunus cerasus), strawberry tree (Arbutus unedo); blueberry or European blueberry (Vaccinium cyanococcus or Vaccinium myrtillus or Vaccinium angustifolium), oxycoccus or cranberry (Vaccinium oxycoccos or Vaccinium macrocarpon), wild strawberry or strawberry (Fragaria vesca or Fragaria spp. or Fragaria ananassa), elderberry (Sambucus nigra), and mixtures thereof.

5. The composition according to claim 1, wherein said at least one species of berries is selected from the group consisting of: blueberry or European blueberry (Vaccinium cyanococcus or Vaccinium myrtillus or Vaccinium angustifolium), oxycoccus or cranberry (Vaccinium oxycoccos or Vaccinium macrocarpon), wild strawberry or strawberry (Fragaria vesca or Fragaria spp. or Fragaria ananassa), elderberry (Sambucus nigra), and mixtures thereof.

6. The composition according to claim 1, wherein said polyphenol fraction of said extract of at least one species of berries comprises proanthocyanidins and/or anthocyanins and/or anthocyanidins.

7. The composition according to claim 1, wherein said at least one bacterial strain comprises Bifidobacterium bifidum MIMBb23sg DSM 32708 and Lactobacillus paracasei LPC-S01™ DSM 26760.

8. The composition according to claim 1, wherein said at least one bacterial strain comprises Bifidobacterium bifidum MIMBb23sg DSM 32708 and Lactobacillus paracasei DG® CNCM I-1572.

9. The composition according to claim 1,
wherein said at least one bacterial strain comprises Bifidobacterium bifidum MIMBb23sg DSM 32708 and Lactobacillus paracasei LPC-S01™ DSM 26760 or Lactobacillus paracasei DG® CNCM I-1572, and
wherein said at least one extract of at least one species of berries comprising the polyphenol fraction of said berries is an extract of blueberry or European blueberry (Vaccinium cyanococcus or Vaccinium myrtillus or Vaccinium angustifolium) or oxycoccus or cranberry (Vaccinium oxycoccos or Vaccinium macrocarpon) comprising the polyphenol fraction of said berries.

10. A method comprising
administering to a subject the composition according to claim 1 as a medicament.

11. A method comprising
administering to a subject an effective amount of the composition according to claim 1 in an effective amount for reducing the production of proinflammatory cytokines and/or increasing the production of anti-inflammatory cytokines.

12. The method of claim 11, wherein said composition is administered as an anti-inflammatory agent.

13. The method according to claim 12, wherein said method is for preventive and/or curative treatment of inflammatory gastrointestinal diseases, disorders and/or symptoms selected from the group consisting of: Helicobacter pylori, peptic or gastric ulcer, duodenal ulcer, chronic inflammatory bowel diseases (IBD), Crohn's disease, ulcerative colitis, microscopic colitis, diverticular disease and diverticulitis.

14. A method comprising
administering to a subject the composition according to claim 1, in an effective amount for preventive and/or curative treatment of musculoskeletal inflammatory diseases, rheumatological diseases, inflammatory articular and/or post-surgery inflammatory diseases.

15. The method according to claim 14, wherein said musculoskeletal inflammatory diseases, rheumatological diseases, inflammatory articular and/or post-surgery inflammatory diseases comprise osteoarthritis, rheumatoid arthritis and/or ankylosing spondylitis.

16. The method according to claim 14, wherein said composition is administered as immunomodulatory and/or immunostimulatory agent capable of reducing the production of IL12 and/or TNF-α cytokines and/or of increasing the production of IL10 cytokines.

17. The method according to claim 14, wherein said musculoskeletal inflammatory diseases, rheumatological diseases, inflammatory articular and/or post-surgery inflammatory diseases comprise osteoarthritis of the joints and/or osteoarthritis of the knee.

18. The composition according to claim 1, wherein said at least one species of berries is selected from the group consisting of blueberry, cranberry, and a mixture thereof.

19. The composition according to claim 1, wherein said at least one bacterial strain consists of Bifidobacterium bifidum MIMBb23sg DSM 32708 and Lactobacillus paracasei LPC-S01™ DSM 26760.

20. The composition according to claim 1, wherein said at least one bacterial strain consists of Bifidobacterium bifidum MIMBb23sg DSM 32708 and Lactobacillus paracasei DG® CNCM I-1572.
